(19) United States
(12) Patent Application Publication
(10) Pub. No.: US 2004/0191297 A1
(43) Pub. Date: Sep. 30, 2004

(54) Carotenoid formulation

(76) Inventors: Lance Schlipalius, Ashwood (AU); Michael Strahan, Blackburn (AU); David X. Healey, Highton (AU); Ernest G. Szoke, King of Prussia, PA (US)

Correspondence Address: COGNIS CORPORATION, PATENT DEPARTMENT, 300 BROOKSIDE AVENUE, AMBLER, PA, 19002, US

(21) Appl. No.: 10/659,475

(22) Filed: Sep. 10, 2003

Related U.S. Application Data
(63) Continuation of application No. 09/446,397, filed on Jun. 22, 2000, now abandoned.
Application No. 09/446,397 is a 371 of international application No. PCT/US98/10356, filed on May 20, 1998.

(30) Foreign Application Priority Data
May 22, 1997 (AU) PO 6933

(51) Int. Cl. A61K 47/00

(52) U.S. Cl. 424/439

(57) ABSTRACT

Compositions comprising lycopene suspended or dispersed in a medium chain triglyceride are described. The medium chain triglyceride may be derived from the esterification of a medium chain fatty acid and glycerol.

### FIELD OF THE INVENTION

The invention relates to a formulation of a carotenoid composition or a carotenoid dispersed in an oil derived from the esterification of fatty acids and glycerol. More particularly, the invention relates to a formulation of a carotenoid composition or a carotenoid in a medium chain triglyceride oil.

### BACKGROUND OF THE INVENTION

There has been a recent growth in the interest of both researchers and industry in a group of compounds referred to collectively as carotenoids. These occur naturally in plants and animals over a wide base and are generally colored, contributing most of the yellow to red colors in fruits and vegetables. The red of crustaceans like lobsters and the cream color of dairy cream is also due to carotenoids. Epidemiological studies suggest that the carotenoid lycopene (a significant dietary source of lycopene is tomatoes) may exert anti-cancer effects in humans. However, as of the present, there have been no studies on the ability of dietary lycopene to inhibit the development of tumors in established animal models.

Due to their high color strength at low concentrations and the high cost of extraction from natural sources, organic chemists successfully synthesized a few carotenoids of economic importance around the middle of this century. Beta-carotene and canthaxathin as well as a few derivatives became commercial products as food colors, dietary supplements and animal product color precursors, notably from the European companies Hoffman-LaRoche AG and BASF AG.

The carotenoids have a unique chemical structure with a backbone of conjugated carbon to carbon double bonds. There are nine double bonds in the main commercial carotenoids which convey special physical and chemical properties to the molecules. In particular, the carotenoids are very reactive with oxygen and free radicals, hence the carotenoids are classified with the traditional anti-oxidants. The carotenoids are present in human, animal and plant tissues. They also are produced in higher concentrations in some living things in response to stress conditions in the environment, such as higher than normal temperatures and high light intensity. Generally they have very low solubilities in water, so they are situated in living cells in association with membranes and lipid aggregates. In living things they are normally present in relatively low concentrations of approximately 10 to 100 ppm.

In the production of higher strength commercial carotenoid products, which are available now from the extraction of natural sources as well as synthetic chemical processes, it is necessary to stabilize the carotenoids from oxidation by oxygen in the atmosphere. There is also degradation of the carotenoid molecules by enzymic or microbiological action, but this normally occurs in lower concentrations (for example, less than 0.1%) and in aqueous environments. Naturally the rate of oxidation varies to a large degree with the chemical structure of the carotenoids. Some are extremely reactive and will spontaneously ignite in air in the finely dispersed high concentration crystalline form. Lycopene is one of the most readily oxidized of the common carotenoids and the stabilization of this material or compositions containing this material is of strategic importance in the production of commercial products. The inability to prevent certain of the carotenoids from oxidizing has rendered their comm rcialization difficult.

Traditionally, stabilization of carotenoids has proven very difficult. One means of helping to prevent oxidation has been to keep the pure crystalline carotenoid materials under an inert gas atmosphere, however, this is only practical on a laboratory scale and not for commercial use. It is also known to suspend carotenoid compositions in triglyceride oils (e.g., soya bean oil) in order to assist in the prevention of oxidation. While the use of standard soya bean oil extracted using solvents and soya beans (which oil has a carbon chain length of mainly between C₁₆ to C₁₈) has proven very effective for some carotenoids such as β-carotene, it has not prevented commercially significant degradation of other carotenoids such as lycopene.

### SUMMARY OF THE INVENTION

The present invention therefore aims to provide a further means to resist the oxidation of carotenoids. Surprisingly, it has been found that the suspension or dispersion of carotenoid compositions or a carotenoid in oils that are derived from the esterification of fatty acids and glycerol (rather than using oils extracted from sources by the use of solvents) decreases the oxidation of carotenoids. Additionally, it has been found that the suspension or dispersion of carotenoid compositions or carotenoid in oils with a chain length of between C₆ to C₁₂ (hereinafter referred to as “medium chain triglyceride oils”) has resulted in a significant decrease in the oxidation of carotenoids.

### BRIEF DESCRIPTION OF THE DRAWINGS

FIG. 1 illustrates the stability of the two compositions described in Example 1 (average for each of the vials tested).

FIG. 2 depicts the time line for the ten-week study described in Example 2 to determine the maximal tolerated dose (MTD) for lycopene in both male (colon model) and female (breast model) rats.

FIG. 3 shows HPLC data for echinenone and lycopene.

FIG. 4 shows HPLC data for all five standard carotenoids, i.e., zeaxanthin, canthoxanthine, echinenone, lycopene, and β-carotene.

FIG. 5 is a standard curve for lycopene generated using crystalline lycopene and echinenone.

FIG. 6 shows the results of HPLC analysis of the Lycopene Suspension.

FIG. 7 shows the chemical structures of carotenoids present in the Lycopene Suspension.

FIGS. 8-11 illustrate the effects of lycopene on animal weight gain.

FIG. 12 shows the results of HPLC-MS analysis of saponified organic extracts from livers which indicates the presence of significant amounts of lycopene, phytofluene and phytoene and their geometrical isomers.

### DETAILED DESCRIPTION OF THE PREFERRED EMBODIMENTS OF THE INVENTION

According to one form of the invention there is provided a formulation including a suspension/dispersion of a carotenoid composition or carotenoid in an oil wherein the oil is derived from the esterification of fatty acids and glycerol. The use of an oil that has been esterified in this way substantially reduces the possibility of introducing into the formulation impurities which are present in oils extracted from sources such as soya bean using solvents. Preferably, substantially pure fatty acids and glycerol are used in the esterification. The fatty acids are preferably purified by distillation. The oil is preferably a medium chain triglyceride oil. Triglycerides are esters of fatty acids and glycerol. The chief constituents of fats and oils, triglycerides have the general formula CH₂(OOCR₁)CH(OOCR₂)CH₂(OOCR₃), wherein R₁, R₂, and R₃ may be of different chain length. In the context of the present invention, preferred triglycerides will have a chain length of C₆ to C₁₂, more preferably C₈ to C₁₀.

According to another form of the present invention, there is provided a formulation including a suspension/dispersion of a carotenoid composition or carotenoid in a medium chain triglyceride oil. Again preferably, the medium chain triglyceride oil has a chain length of C₈ to C₁₀. It is preferred that the medium chain triglyceride oil is esterified from fatty acids and glycerol. Again, substantially pure fatty acids and glycerol are used in the esterification, with the fatty acids preferably purified by distillation.

In a preferred form of the invention, the carotenoid composition predominantly includes carotenoid. Preferably, the carotenoid is selected from the group including lycopene, beta-carotene, zeta-carotene and phtyofluene or mixtures thereof. Lycopene is the preferred carotenoid. Preferably, the carotenoid composition and carotenoid are derived from natural sources.

In a preferred embodiment of the present invention, the medium chain triglyceride oil is the commercially available Delios V™, a medium chain triglyceride oil produced by Grunan Lebensmitteltechnologie. Another preferred medium chain triglyceride oil is registered as CAS 73398-61-5, a mixed glycerin triester with caprylic and capric acid. This medium chain triglyceride oil is available from the Stepan Co., Northfield, Ill., under its trademark Neobee®. Preferably, the acid monomers of the medium chain triglyceride oils are selected from the group including C₆ caproic acid, C₈ caprylic acid, C₁₀ capric acid and C₁₂ lauric acid or mixtures thereof. It is preferable to use purified fatty acids and glycerol to form the medium chain triglyceride oils as the use of these materials decreases the possibility of the oil containing any impurities that may also cause oxidation of the carotenoid. In a preferred form, the medium chain triglyceride oils are derived from natural sources.

In another preferred form of the present invention, the carotenoid comprises up to 10% by weight of the formulation. Preferably, the carotenoid comprises between 4% and 5% by weight of the formulation. In yet another preferred embodiment of the invention, the oil comprises up to 98% by weight, more preferably between 20% and 80% by weight and most preferably between 30% and 70% of the formulation.

Preferably, the composition includes an oil soluble anti-oxidant. The addition of an anti-oxidant further assists in preventing the oxidation of the carotenoid. The anti-oxidants are preferably selected from the group including tocopherols, butylated hydroxy anisole, butylated hydroxytoluene, propyl gallate, ethoxyquin and ascorbyl palmitate plus other natural antioxidant extracts which may be derived from herbs and preferably natural tocopherols, and mixtures thereof. Preferably, the tocopherol is selected from the group including beta, gamma/- and delta-tocopherols or mixtures thereof. In other preferred forms of the invention, the anti-oxidant comprises up to 15% by weight and preferably between 5% and 10% by weight of the formulation.

For oral administration, the composition of the present invention optionally further comprises a pharmaceutically acceptable carrier suitable for oral administration. The oral compositions of the present invention can be made by conventional compounding procedures known in the pharmaceutical art, that is, by mixing the active substances with edible pharmaceutically acceptable non-toxic inert, solid or liquid carriers and/or excipients suitable for systemic administration and conventionally used in oral dosage forms. The pharmaceutical compositions for oral administration may be in the form of tablets, including sustained release forms, lozenges, chewing gum, and capsules. The soft gelatin capsule dosage form is most preferred. These dosage forms may be prepared by those skilled in the art in accordance with known techniques in the art, for example, as described in Remington's Pharmaceutical Sciences, 18th Edition (1990), Mack Publishing Co., Easton, Pa.

The present invention also provides a method of providing a bioavailable antioxidant comprising orally administering to a host an effective amount of lycopene suspended or dispersed in a medium chain triglyceride. In the context of the present invention, the term “host” will be understood to encompass not only humans but also nonhuman animals, particularly mammals. The method comprises administering to the patient an effective amount of a composition comprising a suspension/dispersion of a carotenoid in an oil, such that the dose of carotenoid is between about 1 and 1000 μg/kg. A preferred dose is from about 10 to 100 μg/kg of carotenoid. In practicing this method, a composition according to the present invention may be administered in a single daily dose or in multiple doses per day. The treatment regime may require administration over extended periods of time. The amount per administered dose or the total amount administered will be determined by the physician and depend on such factors as the nature and severity of the disease, the age and general health of the patient and the tolerance of the patient to the compound.

The present invention also provides a method for treating or preventing cancer in a patient in need of such treatment. In the context of the present invention, the term “patient” will be understood to encompass not only humans but also nonhuman animals, particularly mammals. The method comprises administering to the patient an effective amount of a composition comprising a suspension/dispersion of a carotenoid in an oil, such that the dose of carotenoid is between about 1 and 1000 μg/kg. A preferred dose is from about 10 to 100 μg/kg of carotenoid. In practicing this method, a composition according to the present invention may be administered in a single daily dose or in multiple doses per day. The treatment regime may require administration over extended periods of time. The amount per administered dose or the total amount administered will be determined by the physician and depend on such factors as the nature and severity of the disease, the age and general health of the patient and the tolerance of the patient to the compound.

This method may be practiced by administration of a composition according to the present invention by itself, or in combination with other active ingredients, including other antioxidants, and/or therapeutic agents in a pharmaceutical composition. Other therapeutic agents suitable for use herein are any compatible drugs that are effective by the same or other mechanisms for the intended purpose, or drugs whose actions are complementary to or synergistic with those of the present formulations or compositions.

The compounds or agents utilized in combination therapy may be administered simultaneously, in either separate or combined formulations, or at different times than the compositions according to the present invention, e.g., sequentially, such that a combined effect is achieved. The amounts and regime of administration will be adjusted by the practitioner, by preferably initially lowering their standard doses and then titrating the results obtained.

With the foregoing and other objects, advantages and features of the invention that will become hereinafter apparent, the nature of the invention is further explained in the following detailed description of the preferred embodiments of the invention and in the appended claims.

### EXAMPLES

### Example 1

### Formulation and Stability Testing

In order to demonstrate the improved anti-oxidant effect of the esterified oil, two comparative experiments were conducted using a carotenoid composition which included lycopene as the carotenoid (“the Lycopene Composition”). One experiment involved suspending/dispersing the Lycopene Composition in soya bean oil extracted using solvents from soya bean and the other involved suspending/dispersing the Lycopene Composition in a medium chain triglyceride oil esterified from fatty acids and glycerol. The results were then compared.

Materials

The lycopene composition used in the experiments was extracted from tomatoes.

The medium chain triglyceride oil used was manufactured by fractionation of the fatty acids of coconut oil and the esterification of the selected fraction of eight and ten carbon fatty acids with high purity glycerol. Forming the oil in this way may reduce the impurities that appear in oils extracted directly from plant and animal sources. The soya bean oil was a standard oil that had been attracted from soya beans.

Two portions of 10 kilograms of the lycopene composition were each dispersed in 15 kilograms of each of the soya bean oil (“Composition 1”) and the medium chain triglyceride oil (“Composition 2”) as a diluent or suspending/dispersing oil to form a continuous phase by blending using an efficient mixing procedure. One gram samples of each composition were placed in glass vials for testing.

Test Procedures and Results

The concentrations of lycopene in each of Composition 1 and Composition 2 were measured using a spectrophotometer to determine the concentration of lycopene at the following times:

0 Month (i.e., shortly after forming the Compositions);

1 Month;

2 Months; and

3 Months.

(a) Analytical Method

Each sample was dissolved in chloroform and diluted to a suitable concentration with cyclohexane. Absorbance was measured at specific wavelengths at 20° C., and the concentration was calculated from a known extinction coefficient.

The following reagents and equipment were used:

Spectrophotomet r with 10 mm glass cells

Analytical balance

100 ml volumetric flasks

50 ml volumetric flasks

2.0 ml bulb pipettes

Chloroform AR grade

Cyclohexane

In carrying out the testing, because lycopene is degraded slowly in light, low actinic glass was used. For each sample, the following steps were followed. 0.5 to 1.0 g of 5% lycopene in each of the oils were separately weighed accurately into a 100 ml volumetric flask. Approximately 5 ml of chloroform was added and the reagents were mixed well to dissolve the samples.

Each sample was viewed against light to ensure that it was completely dissolved.

To ensure that each sample was completely dissolved, it was allowed to stand for 5 minutes and 5 ml additional chloroform was added and warmed under hot tap water (as necessary).

The solution was diluted to volume with cyclohexane and mixed well. This was solution A. 2 ml of solution A was pipetted into a 50 ml volumetric flask and diluted to volume with cyclohexane and mixed well. This was solution B.

2 ml of solution B was pipetted into a 50 ml volumetric flask and diluted to volume with cyclohexane and mixed well; this was solution C.

A spectrophotometer (which had a 2 nm slit width) was zeroed using cyclohexane in both cells and the absorbance of solution C was measured at 472 nm against a cyclohexane blank. This analysis was conducted in duplicate.

In calculating the results, the following standards were used:

A=Absorbance of solution C at 472 nm

m=Weight of sample

E=Extinction coefficient

$$E \frac{1 \%}{1 cm}$$

at 472 nm=3450

C=Concentration of lycopene (%)

$$C = \frac{A \times Dilution}{E \times m}$$

In the interval between the testing periods both compositions were stored at 25° Celsius.

It was found during, testing that lycopene tended to form large crystals, in some cases 2 to 3 mm in size, over a period of months in storage. As the formation of large crystals can substantially influence the assay result, due to sampling error, the one gram samples of the material in glass vials were used which had been prepared immediately after production and stored for stability tests at 25° Celsius. At monthly intervals, vials were removed for assay and the complete contents assayed to overcome the sampling problem due to crystallization.

(b) Results

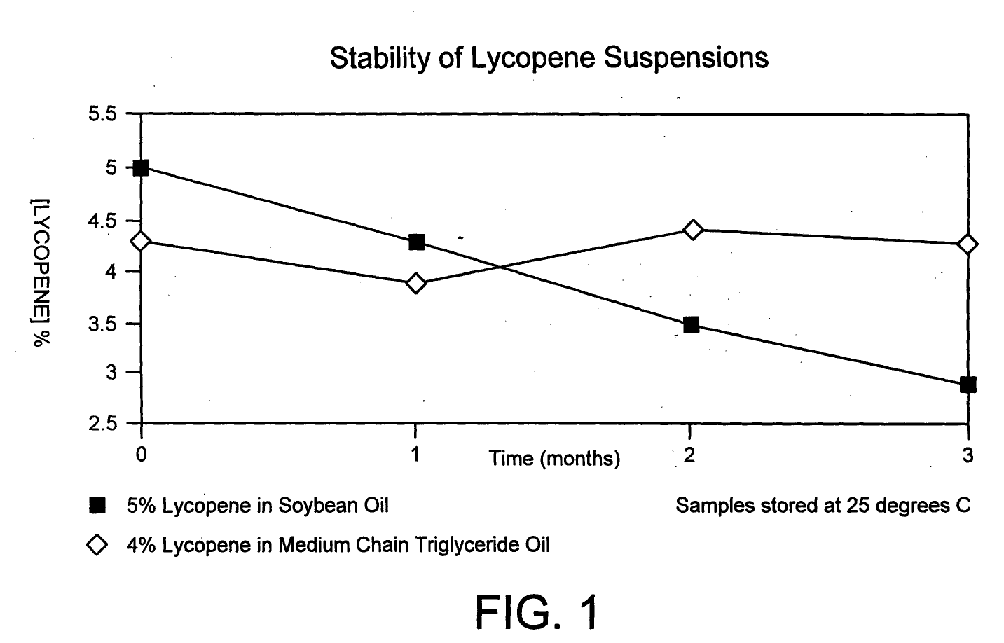

The results of the stability of the two Compositions were averaged for each of the vials tested, as shown in FIG. 1 and Table 1:

TABLE 1

| | Stored at 25 degrees Celsius | | | |
|---|---|---|---|---|
| | | | 2 | 3 |
| TIME FROM PRODUCTION | 0 month | 1 month | months | months |

COMPOSITION 1: LYCOPENE IN SOYA BEAN OIL

| | | | | |
|---|---|---|---|---|
| LYCOPENE CONTENT % w/w | 5.0 | 4.3 | 3.5 | 2.9 |

COMPOSITION 2: LYCOPENE IN MEDIUM CHAIN
TRIGLYCERIDE OIL

| | | | | |
|---|---|---|---|---|
| LYCOPENE CONTENT % w/w | 4.3 | 3.9 | 4.4 | 4.3 |

Discussion of Results

The results of the above experiments show that over a three month period, the lycopene concentration in the medium chain triglyceride oil (Composition 2) was relatively constant and in fact the average percentage concentration after three months was the same as the concentration taken immediately after Composition 2 was prepared. The results of the concentration of lycopene in the soya bean oil (Composition 1) shows a steady decline in the % weight of the lycopene over a three month period.

The results demonstrate that the oil derived from the esterification of fatty acids and glycerol (in this case a medium chain triglyceride oil) had a stabilizing effect on the oxidation of lycopene.

### Example 2

### Preclinical Toxicity and Stability of Lycopene

The purpose of these experiments was to determine the biologically acceptable dose range, uptake and tissue disposition of dietary lycopene.

Specific Aims

To determine the maximum tolerated dose of lycopene administered in the AIN-76A diet, in male and female Fischer 344 rats and to assess the uptake and disposition of dietary lycopene in blood, feces and various organs such as mammary gland, prostate, colon and lung.

Methods

Inbred Fischer F344 males (n=70) and females (n=70) were obtained from Taconic Farms, Germantown, N.Y. At about 35 days of age, all animals were transferred to the experimental room from quarantine. The animals were then allocated into experimental groups using formal randomization methods to ensure that each group contained animals of the same mean weight.

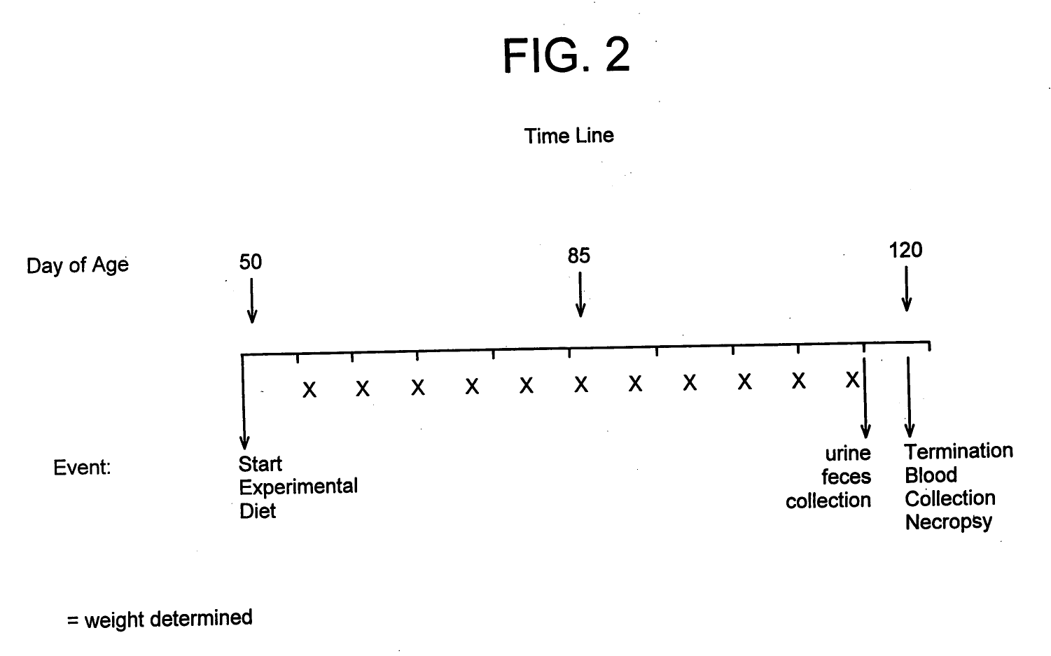

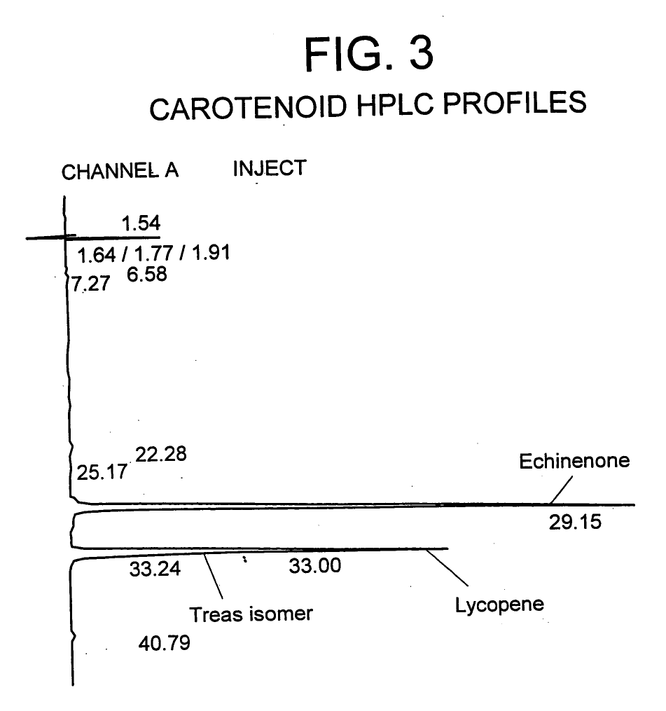

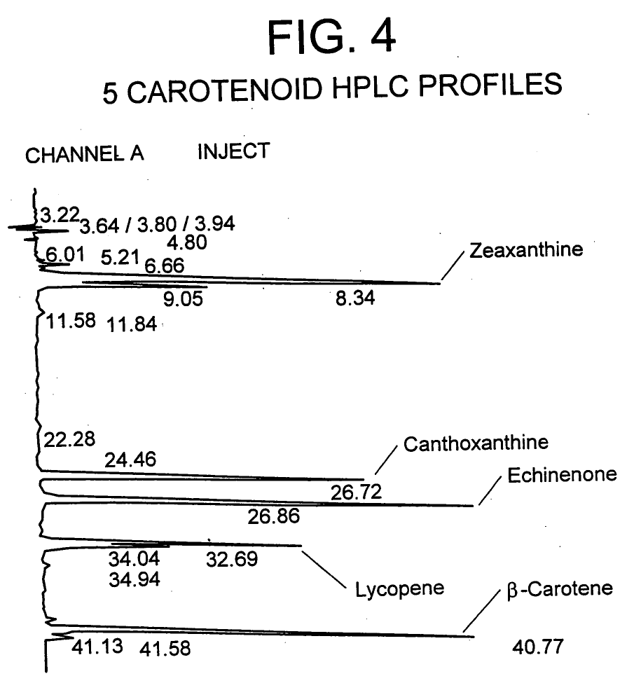

A ten-week pilot study was conducted as described in Table 2 & FIG. 2 to determine the maximal tolerated dose (MTD) for lycopene in both male (colon model) and female (breast model) rats. The choice of dose ranges was based on published values for β-carotene. A mixed carotenoid composition containing 5.7% carotenoids, including 3.7% pure lycopene was suspended in medium chain triglycerides (this composition will be referred to below as the “Lycopene Suspension”). The Lycopene Suspension was incorporated into the AIN-76A diet.

TABLE 2
MTD STUDY

| | | | Lycopene quantity^{a} | | |
|---|---|---|---|---|---|
| No Animals | | | (mM/kg | Estimated | Estimated |
| Group | Males | Females | diet) | (mg/kg diet) (ppm) | (% diet) |
| 1 | 10 | 10 | 2.5 | 1280 | .128 |
| 2 | 10 | 10 | 1.0 | 512 | .0512 |
| 3 | 10 | 10 | 0.5 | 256 | .0256 |
| 4 | 10 | 10 | 0.25 | 128 | .0128 |
| 5 | 10 | 10 | 0.10 | 51 | .0051 |
| 6 | 20 | 20 | 0 | 0 | 0 |
| Total N | 70 | 70 | | | |

^{a}Based on Lycopene MW = 536.

The diets were formulated under reduced light and stored at −4° C. in air-tight containers in 4 kg lots. The components of the AIN-76A diet are listed in Table 3.

TABLE 3
STANDARD AIN-76A DIET

| INGREDIENT | QUANTITY (g/100 g) |
|---|---|
| Casein | 20 |
| Corn starch | 52 |
| Dextrose | 13 |
| Corn oil | 5 |
| DL-Methionine | 0.3 |
| Choline Bitartrate | 0.2 |
| Alphacel | 5 |
| AIN-76 Vitamin mix | 1.0 |
| AIN-76 Mineral mix | 3.5 |
| Total | 100 |
| Energy Value (kCal/g) | 3.89 |

Differences in the lipid content of the various treatment groups were controlled by supplementing their diets with appropriate amounts of Neobee® medium chain triglyceride (Stepan Co., Maywood, N.J.) such that all animals, including controls, received the same amount of dietary fat.

Prior to termination, 3 animals from each group were placed in metabolic cages and 24-hour urine and fecal collections made for subsequent lycopene assay. Blood was collected by heart puncture under anesthesia for lycopene assay and all animals were sacrificed by CO₂ anesthesia. Blood and feces were stored at −20° C. At necropsy, all major organ systems were grossly inspected. Any unusual appearing tissues were placed in formalin and saved for later histological examination.

Animal weights were recorded once per week and animal appearance (coat texture) and food avoidance behavior assessed. By concentration,

TABLE 4
HPLC METHOD FOR LYCOPENE ASSAY

| | |
|---|---|
| Pump | Waters Model 510 |
| Detector | Shimadzu, SPD-10A UV-Vis Detector |
| Integrator | Waters 745 Data Model |
| Controller | Waters Automated Gradient Controller |
| Column | Rainin Microsorb 5 μm, C18, 25 cm × 4.6 mm |
| Wavelength | 470 nm |
| Rate | 0.7 ml/min |
| AUFS | 0.032 |
| Chart Speed | 0.25 cm/min |
| Volume of Injection | 20-50 μl |
| Solvent A | 90% Acetonitrile |
| | 10% Methanol |
| Solvent B | 45% Hexane |
| | 45% Methylene Chloride |
| | 10% Methanol |
| | for every 500 ml solvent B, add 0.5 ml |
| | Diisopropylethylamine |

Program

| Time | Solvent A % | Solvent B % |
|---|---|---|
| 0 | 95 | 5 |
| 10 | 95 | 5 |
| 40 | 55 | 45 |
| 45 | 95 | 5 |
| 65 | 95 | 5 |

Injection Solvent Mixture
40% Acetonitrile
20% Methanol
20% Hexane
20% Methylene Chloride

the maximal tolerated dose is that dose which results in >10% mean body weight loss.

Quantitative Determination of Lycopene by HPLC

The HPLC equipment, solvent systems, and solvent program are outlined in Table 4. Crystalline carotenoids were used as standards including: α-carotene, β-carotene, lycopene, zeaxanthin, cryptoxanthin and echinenone (Hoffmann-LaRoche). Echinenone, which is a ketone derivative of β-carotene, was used as an internal standard. Stock solutions of standards were prepared at a concentration of 30 μg/ml in injection solvent (Table 4).

| | | |
|---|---|---|
| (1) | Lycopene | (RO 01-9251-00) |
| (2) | Zeaxanthin | (RO 01-9371-000) |
| (3) | Cryptoxanthine | (RO 04-0763-000) |
| (4) | 9-Carotene | (RO 01-8300-000) |
| (5) | Echinenone | (RO 04-2847-000) |

The variability and reliability of the system was tested by comparing multiple samples from a standard solution of lycopene and echinenone over a period of 5 days (Table 5). The retention times for lycopene varied from a low of 32.58 to a high of 35.95 minutes; that of echinenone varied from a low of 28.54 to a high of 30.64 minutes. The lycopene/echinenone ratio varied from a low of 1.12 to a high of 1.20. A similar low level of within-sample variation was found when integrated areas under curves were compared (Table 5).

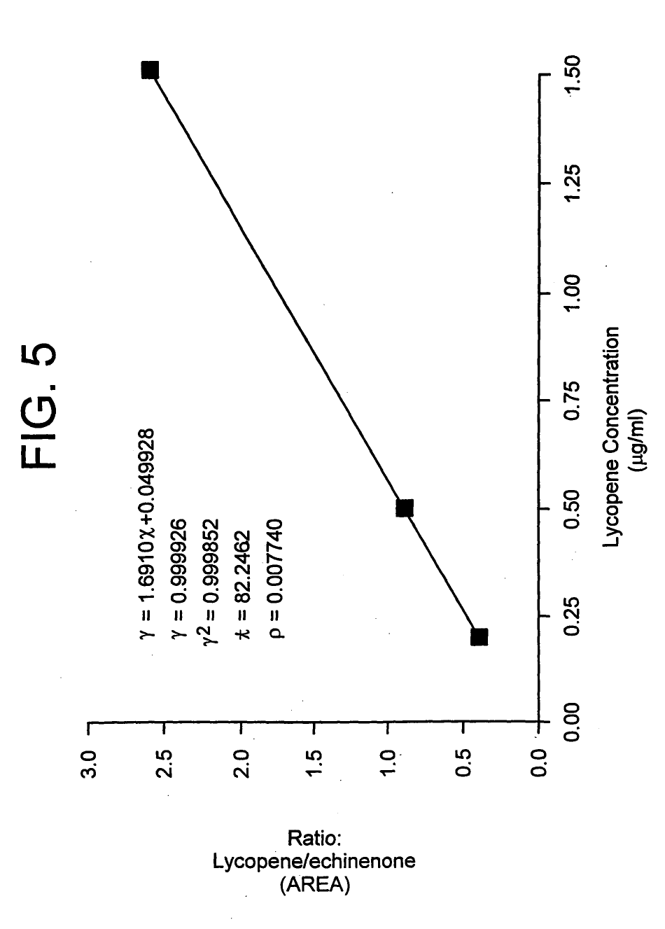

Using crystalline lycopene and echinenone, a standard curve was generated for lycopene (FIG. 5). Experimental values were read from the standard curve and expressed in terms of either ng/ml or ng/μg/gram tissue wet weight.

TABLE 5
CAROTENOID HPLC STUDY: VARIABILITY AND RELIABILITY

| | RT | | | Area | | |
|---|---|---|---|---|---|---|
| Run | Lycopene | Echinenone | Ratio(L/E) | Lycopene | Echinenone | Ratio (L/E) |
| Day 1 | | | | | | |
| 1 | 33.17 | 29.15 | 1.14 | 586,132 | 720,121 | 0.81 |
| 2 | 32.91 | 28.89 | 1.14 | 567,339 | 755,926 | 0.75 |
| 3 | 33.49 | 29.86 | 1.12 | 527,365 | 737,088 | 0.72 |
| 4 | 34.48 | 30.47 | 1.13 | 532,491 | 765,646 | 0.70 |
| 5 | 34.47 | 30.45 | 1.13 | 527,024 | 772,265 | 0.68 |
| 6 | 34.64 | 30.64 | 1.13 | 496,268 | 768,335 | 0.65 |
| Day 3 | | | | | | |
| 7 | 34.05 | 28.30 | 1.20 | 511,111 | 781,883 | 0.65 |
| 8 | 33.04 | 29.03 | 1.14 | 551,826 | 736,851 | 0.75 |
| Day 5 | | | | | | |
| 9 | 32.99 | 28.78 | 1.15 | 624,986 | 803,354 | 0.78 |
| 10 | 32.58 | 28.32 | 1.15 | 573,498 | 793,606 | 0.72 |
| 11 | 32.81 | 28.54 | 1.15 | 562,057 | 823,087 | 0.68 |
| 12 | 35.95 | 30.11 | 1.19 | 612,266 | 859,039 | 0.71 |
| 13 | 34.50 | 29.86 | 1.16 | 543,684 | 869,292 | 0.63 |
| \overline{X} | 33.78 | 29.42 | 1.15 | 555,085 | 783,576 | 0.71 |
| S | 0.99 | 0.85 | 0.023 | 37,924 | 45,566 | 0.053 |
| C.V. (%) | 2.9 | 2.9 | 2.0 | 6.8 | 5.8 | 7.5 |

RT = Retention time in minutes
AREA = Integrated area under curve

Diet

Extraction of lycopene from diet was as follows: A sample of diet containing lycopene was suspended in the injection solvent (Table 4) and homogenized in a polytron homogenizer. Echinenone was added as an internal standard and the mixture centrifuged at 3000 rpm at 4° C. for 15 minutes. The supernatant was decanted and saved and the extraction repeated. The pooled extracts were then dried under N₂ gas at 60° C. and the residue dissolved in 0.5 ml injection solvent and then subjected to HPLC analysis. The same method, with minor variations, was used for feces and for lung tissue. The efficiency of lycopene extraction from the diet is shown in Table 6 A&B. At any concentration tested the efficiency ranged from 75% at the highest concentration to 100% for the lowest concentration of lycopene.

Serum

Extraction of lycopene from serum was conducted by adding 1 ml of serum to 0.1 ml echinenone standard in ethanol and 0.9 ml ethanol. The mixture was then vortexed for 20 seconds, and 2.0 ml hexane added and mixed vigorously for 1 minute. The cloudy mixture was then centrifuged at 3,000 rpm for 15 minutes at 4° C. and the clear hexane layer was decanted. The above procedure was repeated 3-5×. The pooled hexane fraction was then dried under N₂ gas at 60° C., and the residue dissolved in 0.5 ml HPLC solvent and analyzed by HPLC.

Tissue

Breast, liver and prostate tissue contain lipids which interfere with the assay. Consequently, a saponification step was required prior to organic extraction. Tissue was homogenized in 20 ml methanol to which echinenone was added as internal standard. For breast tissue, which comprise of 85% adipocytes, 8.0 ml 50% NaOH and 4.0 ml 25% sodium ascorbate was added and the mixture was homogenized. For prostate and liver tissue, which contain less lipid, 4.0 ml and 50% NaOH. and 2.0 ml sodium ascorbate were added and the mixture was

TABLE 6A & 6B
EFFICIENCY OF LYCOPENE EXTRACTION
FROM THE AIN-76A DIET
6A

| | Lycopene | | | | | |
|---|---|---|---|---|---|---|
| | Con- | | | | Lycopene | |
| | centration | Sample | Volume | Volume | concentration | |
| Group | in diet | weight | of extract | injected | following extraction | |
| # | (μg/g) | (g) | (ml) | (μl) | (μg/g) | |
| 1 | 1,280 | 0.5 | 100 | 20 | 975 | (76%) |
| 2 | 512 | 0.5 | 100 | 20 | 321 | (63%) X |
| | | | | | 403 | (79%) |
| 3 | 256 | 0.5 | 50 | 20 | 192 | (75%) |
| 4 | 128 | 0.5 | 50 | 50 | 122 | (95%) X |
| | | | | | 101 | (86%) |
| 5 | 51 | 1.0 | 50 | 50 | 62 | (100%) X |
| | | | | | 52 | (100%) |

6B

| | Echinenone | Lycopene | Ratio | Concentration |
|---|---|---|---|---|
| No. | Area under curve | | (L/E) | (μg/g) |
| standard | 2,590,806 | 2,191,332 | 0.85 | |
| | 2,754,253 | 2,505,114 | 0.91 | |
| 1 | 2,891,211 | 4,493,499 | 1.55 | 975 |
| 2 | 2,980,758 | 1,527,316 | 0.51 | 321 X |
| | 2,618,266 | 1,663,187 | 0.64 | 403 |
| 3 | 2,804,003 | 1,705,160 | 0.61 | 192 |
| 4 | 2,236,710 | 2,179,322 | 0.97 | 122 X |
| | 2,636,044 | 2,087,983 | 0.80 | 101 |
| 5 | 2,647,478 | 2,620,900 | 0.99 | 62 X |
| | 2,613,385 | 2,171,047 | 0.83 | 52 |

homogenized. Saponification was carried out in 50 ml tubes saturated with N₂ gas overnight in a temperature controlled shaker bath at 30° C. Following saponification, 10 ml hexane was add d to each tube and vortexed for 1 minute. The mixture was then centrifuged at 2000 rpm for 10 min at 4° C. and the hexane layer was decanted and saved. This hexane extraction procedure was repeated 5-7 times. The pooled hexane extract was then dried under N₂ gas at 60° C., dissolved in injection solvent and analyzed by HPLC. Lung and colon tissue were processed in an identical manner with the exception that the saponification step was omitted.

I. Analysis of the Lycopene Suspension

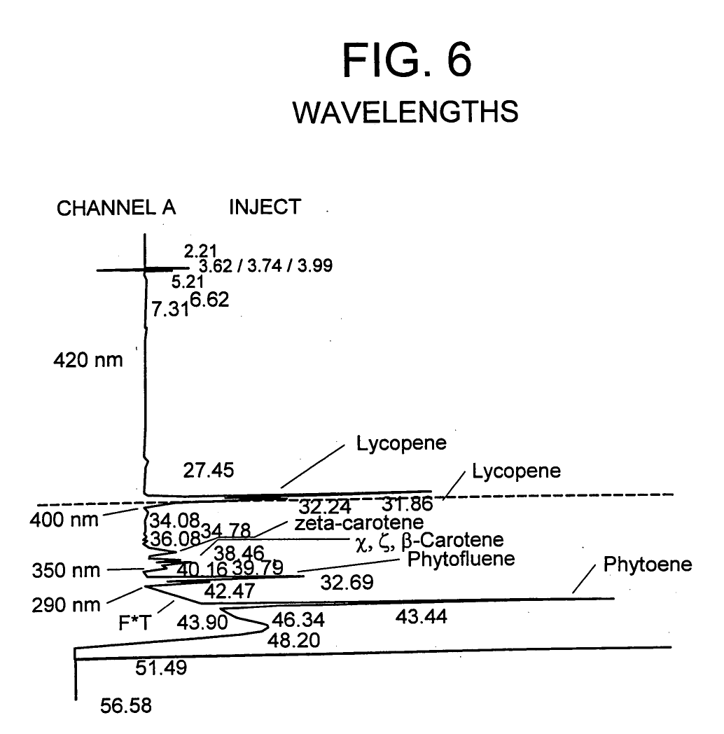

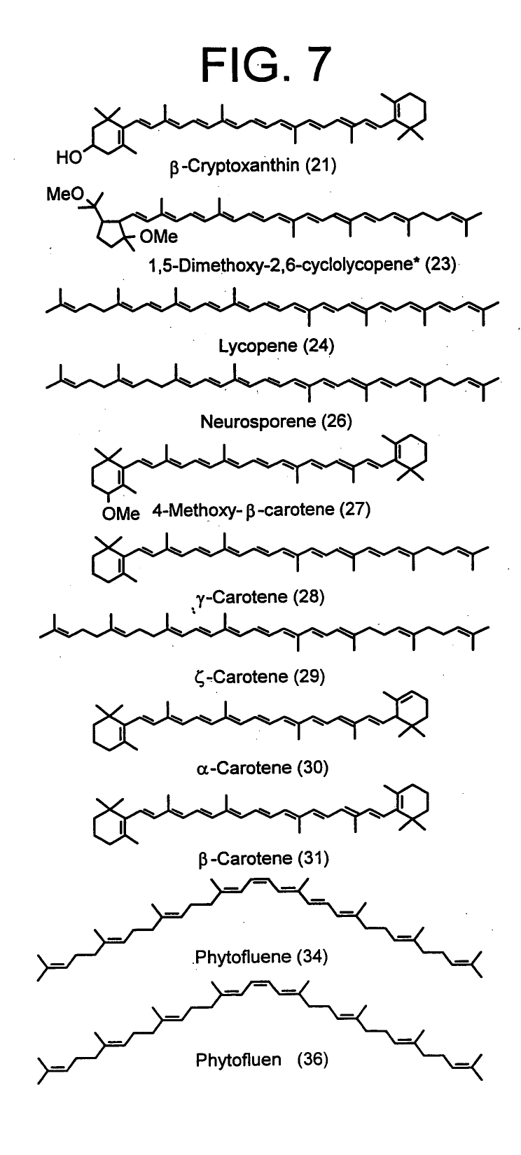

When the Lycopene Suspension (Henkel Corporation, La Grange, Ill.) was extracted by organic extraction methods, the major carotenoid was lycopene, but a number of other carotenoids were known to be present in tomatoes, α- and β-Carotene, phytofluene and phytoene were also present (FIGS. 6 & 7). The quantitative profile of carotenoids in the Lycopene Suspension was carried out using an HPLC system as described above, equipped with a photodiode array detector. The six major components can be seen in Table 7.

TABLE 7
QUANTITATIVE CAROTENOID PROFILE
OF THE LYCOPENE SUSPENSION

| | μg/gm | % (g/100 g) | % of total carotenoid |
|---|---|---|---|
| 1. Lycopene | 37,504 | 3.7504 | 66 |
| 2. β-Carotene | 12,443 | 1.2443 | 22 |
| 3. Phytofluene | 3,349 | 0.3349 | 6 |
| 4. Phytoene | 2,794 | 0.2794 | 5 |
| 5. zeta-Carotene | 440 | 0.0440 | 0.7 |
| 6. 2,6 cyclolycopene- | 471 | 0.0440 | 0.7 |
| 1,5-diol | | | |
| Total Carotenoids | 57,001 | 5.7 | 100 |

Lycopene is present at 3.7%, however, the sum of all the carotenoids is approximately 5.7%. As a consequence, when expressed in terms of % total carotenoids, lycopene comprises 67% of the total, β-carotene 20%; the lycopene precursers, phytoflourene and phytoene, 5% each, and z-carotene and 2,6-cyclolycopene-1,5-diol 0.7%. Recalculating the lycopene concentration in the diet based on a 5.72% (rather than 4%) total carotenoid content (67% of which is lycopene) results in the following concentrations per treatment group: Group 1, 1280; Group 2, 512; Group 3, 256; Group 4, 128; and Group 5, 51 (mgs/kg diet). Hence, rather than a pure lycopene preparation, the Lycopene Suspension contains a 5.9% suspension of lycopene, α- and β-carotene, phytofluene and phytoene (precursors of lycopene) suspended in medium chain triglyceride. This is actually preferable for chemoprevention studies since the carotenoid profile of the Lycopene Suspension is close to that of commercial tomatoes. (See Khachik, F, Beecher, G.R. International Conference on Food Factors: Chemistry and Cancer Prevention. Distribution of carotenoids in fruits and vegetables as a criterion for the selection of appropriate chemopreventive agents. H. Ohigashi (ed.), Springer-Verlag, Tokyo, 1996 (in press).

II Stability in Feed

The stability of the lycopene suspension once incorporated into the diet was assessed in two ways. The first was by placing the diet containing the suspension at 4° C. in the dark for a period of 3 weeks (Table 8A,B).

TABLE 8A & 8B
DIETARY LYCOPENE STABILITY STUDY^{a}
8A

| | | # days | Lycopene concentration | |
|---|---|---|---|---|
| Group # | Date | in refrigerator | (μg/g) | %^{b} |
| 1 | 5/1 | 1 | 748 | 101 |
| 2 | 5/8 | 8 | 728 | 98 |
| 3 | 5/14 | 14 | 672 | 90 |
| 4 | 5/21 | 21 | 534 | 72 |

^{a}AIN-76A diet
^{b}Taking concentration of diet (04/40), 744 μg/g, as 100%

8B

| | | | |
|---|---|---|---|
| Echinenone^{a} | Lycopene | Ratio | Concentration |

| | | | |
|---|---|---|---|
| Group # | Area under curve | (L/E) | (μg/g) |

| | | | | |
|---|---|---|---|---|
| standard | 2,461,649 | 2,117,904 | 0.86 | |
| 1 | 2,304,361 | 2,486,290 | 1.08 | 748 |
| 2 | 2,422,134 | 2,545,973 | 1.05 | 728 |
| 3 | 2,325,379 | 2,265,087 | 0.97 | 672 |
| 4 | 2,334,947 | 1,787,656 | 0.77 | 534 |

^{a}Internal standard

At various time points, a diet sample was taken and lycopene was extracted as follows: Approximately 0.5 to 1.0 g of sample was added to a 15 ml volume of extraction solvent consisting of 40% acetonitrile, 20% hexane, 20% methanol and 20% methylenechloride. The mixture was sonicated for 2 minutes and allowed to stand for 5 minutes. The reddish-orange clear supernatant was then decanted. This process was repeated 4 times, at which time the supernatant was colorless. Solvent was then added to a volume of 25 to 100 ml, the mixture centrifuged, and the supernatant decanted. All supernatants were then mixed. The above solvent serves two purposes; first, it is the most efficient mixture for lycopene, and second, it is the same solvent used for injecting lycopene samples into the HPLC. As seen in Table 8, by 3 weeks lycopene decomposes at 4° C. in the dark to about 72% of the amount present on day 1; by two weeks, about 10% decomposition occurs. This suggested that a two week storage period would be best for the feeding study.

The second was by exposing the lycopene-containing diet to air and light, as is the case in an actual feeding study. The stability of lycopene in the food cup where it is exposed to light and air is seen in Table 9.

TABLE 9
STABILITY OF LYCOPENE INCORPORATED INTO AIN-76A
DIET AT AMBIENT TEMPERATURE

| Date | Days in food cup | Concentration (μg/g) |
|---|---|---|
| 4/30 | 0 | 744 |
| 5/2 | 2 | 702 |
| 5/3 | 3 | 616 |
| 5/6 | 6 | 547 |
| 5/7 | 7 | 439 |
| 5/9 | 9 | 624 |
| 5/10 | 10 | 576 |
| 5/13 | 13 | 449 |
| 5/14 | 14 | 419 |
| 5/15 | 15 | 425 |
| 5/17 | 17 | 431 |
| 5/20 | 20 | 385 |
| 5/21 | 21 | 376 |

It appears that a small amount is lost over a 48-hour period. By 72 hours, approximately 20% of the total lycopene is lost. After 3 weeks, one-half of the original lycopene is no longer measurable. Based on these results, the food was allowed to remain in the food cups for no longer than 48 hours.

An analysis of all the major carotenoids present in the Lycopene Suspension-containing diets after 7 months storage at 20° C. (Table 10). Note that all the major carotenoids plus the lycopene metabolite present in the Lycopene Suspension are present in the diets in the same relative proportions. However, the net loss of lycopene over 7 months was 66%, 38%, 40%, 44% and 60%, in treatment groups 1-5, respectively; in contrast, β-carotene concentrations declined by only 10% of original values over the same time period.

TABLE 10
CAROTENOID CONTENT OF DIETS
SUPPLEMENTED WITH THE LYCOPENE SUSPENSION^{a}

| | | μg/gm (ppm) | | | | | |
|---|---|---|---|---|---|---|---|
| | | | | | | | 2,6 |
| | mM/kg | | β- | zeta- | | | cyclolycopene- |
| Diet | diet | Lycopene | carotene | carotene | Phytofluene | Phytoene | 1,5-diol |
| 1 | 2.5 | 472 | 284 | 1 | 58 | 70 | 2 |
| 2 | 1.0 | 348 | 138 | 9 | 28 | 23 | 1.4 |
| 3 | 0.5 | 168 | 69 | 4 | 14 | 0.32 | 0.76 |
| 4 | 0.25 | 92 | 37 | 2 | 8 | 5 | 0.46 |
| 5 | 0.10 | 22 | 8 | 0 | 2 | 1 | 0.14 |
| 6 | 0 | 0 | 0 | 0 | 0 | 0 | 0 |

^{a}Diets stored at −20° C. for 7 months

III. Preliminary Feeding Study

One male rat was fed 2.5 mM lycopene (+other carotenoids)/kg diet for a period of 3 weeks. At this high dose, no change in body weight or food consumption was seen. The feces exhibited a distinct red aspect, as did parts of the coat. The tail was brown. At necropsy, the liver was a deep red, as was the cecum and small intestine. Little discoloration was seen in the abdominal adipose tissue or in the prostate and seminal vesicle.

Analysis of the 24 hour fecal pellets for lycopene (Table 11) indicated that approximately 55% of total dietary lycopene consumed was excreted in the feces. This suggests that the remainder was absorbed in the gut. The possibility that some lycopene decomposed upon storage prior to extraction must also be considered. It was found that freeze-drying the fecal pellets prior to lycopene extraction was the most efficacious method for extracting lycopene. This, no doubt, is due to the presence of water in the fecal pellet and to the geometry of the pellet. Once freeze-dried, the fecal pellet can easily be powdered, permitting easier access of the solvent to the fecal matrix.

TABLE 11
FECAL LYCOPENE ANALYSIS

| | | | Estimated | | | 24 hr | | |
|---|---|---|---|---|---|---|---|---|
| | Lycopene | Diet | total | 24 hr fecal | Fecal | fecal | | |
| | in diet | Consumed | lycopene | weight | lycopene | lycopene | Output/ | % |
| | (μg/g) | (g/24 h) | (μg) | (g) | (μg/g) | (μg) | input | Efficiency |
| Organic | 744 | 13-15 | 9,784 | 1.44 | 2,992 | 4,309 | 4,309 | 44 |
| Extraction | | | | | | | 9,784 | |
| Lypholization + organic | 744 | 13-15 | 9,784 | 1.44 | 3,754 | 5,406 | 5,405 | 55 |
| extraction | | | | | | | 9,784 | |

IV. Feeding Study

(1) Weight Gain

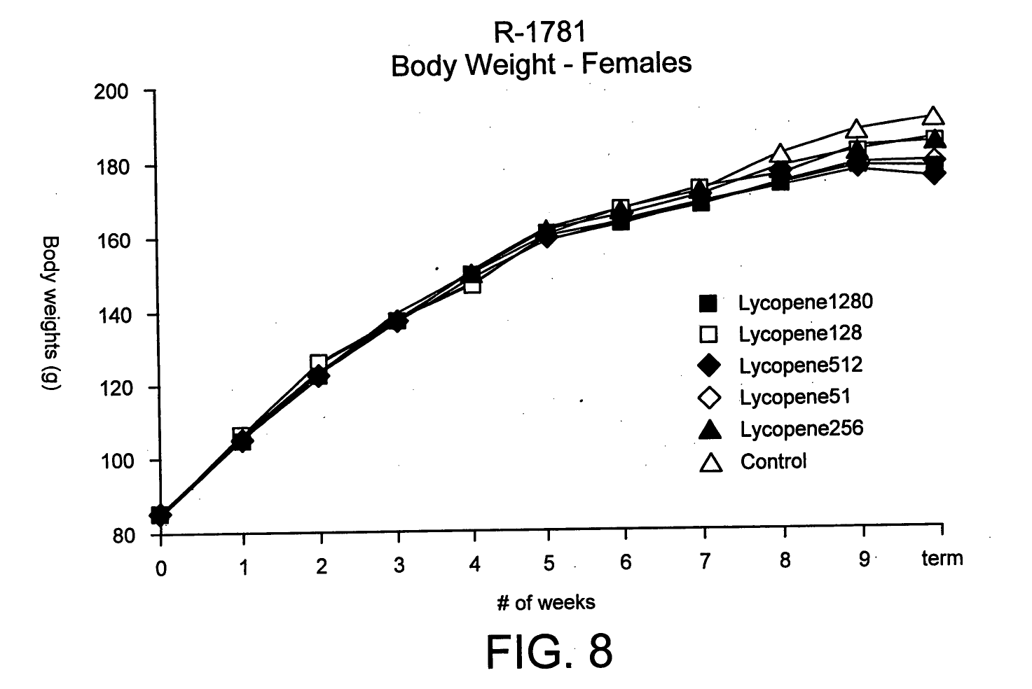

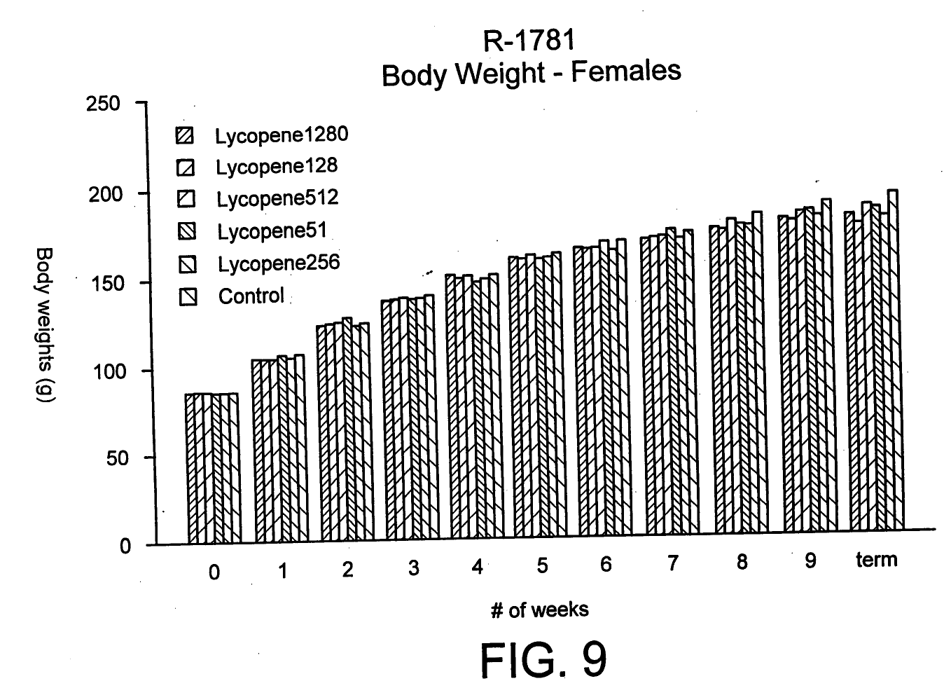

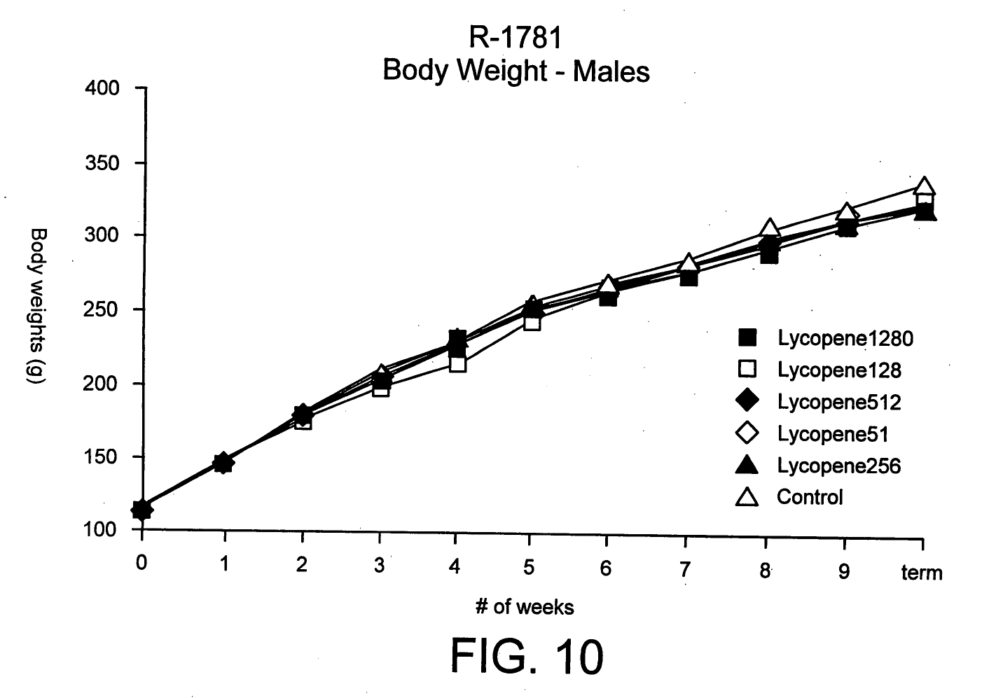

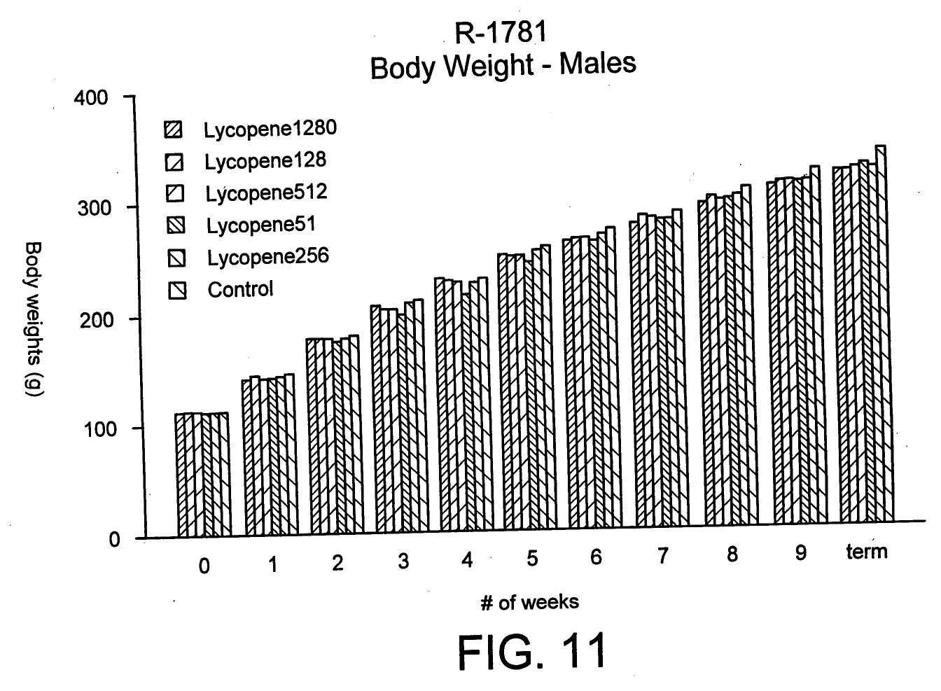

The effects of lycopene on animal weight gain can be seen in Tables 12 & 13, FIGS. 8-11. There was no difference in the weight gains of rats fed diets supplemented with different doses of lycopene when assessed by analysis of variance for repeated measures. There was an indication by the end of 10 weeks that control rats were gaining weight to a slightly greater extent than supplemented rats, but this difference did not exceed 10%, the usual cut-off point indicating food avoidance or toxicity.

TABLE 12
EFFECT OF DIETARY LYCOPENE ON WEIGHT
GAIN IN FEMALE F344 RATS^{a}

weight (g × SD)

| Group | Lycopene mixture (ppm) | Week 0 | Week 8 |
|---|---|---|---|
| 1 | 1280 | 86 ± 5 | 168 ± 10 |
| 2 | 521 | 86 ± 5 | 169 ± 9 |
| 3 | 251 | 86 ± 5 | 171 ± 13 |
| 4 | 128 | 86 ± 5 | 173 ± 8 |
| 5 | 51 | 86 ± 5 | 168 ± 13 |
| 6 | Controls | 86 ± 5 | 172 ± 15 |

^{a}N = 10/group

TABLE 13
EFFECT OF DIETARY LYCOPENE ON WEIGHT
GAIN IN MALE F344 RATS^{a}

weight (gms × SD)

| Group | Lycopene mixture (ppm) | Week 0 | Week 8 |
|---|---|---|---|
| 1 | 1280 | 113 ± 6 | 276 ± 30 |
| 2 | 512 | 113 ± 6 | 280 ± 19 |
| 3 | 256 | 113 ± 6 | 279 ± 26 |
| 4 | 128 | 113 ± 6 | 279 ± 12 |
| 5 | 51 | 113 ± 6 | 280 ± 22 |
| 6 | Controls | 113 ± 6 | 285 ± 21 |

^{a}N = 10/group

(2) Tissue and Serum Uptake

Liver

Lycopene was concentrated to a 100-1000 fold greater extent in the liver compared to serum or other organs. Hepatic lycopene levels ranged between 33-120 μg/gm wet weight at the highest dose of lycopene 80 ppm) (Table 14).

TABLE 14
CAROTENOID CONTENT OF LIVERS OF RATS FED
DIETS SUPPLEMENTED WITH THE LYCOPENE SUSPENSION^{a}

| | | μg/gm (ppm) | | | | | |
|---|---|---|---|---|---|---|---|
| | mM/kg | | | zeta- | | | 2,6 cyclolycopene- |
| Diet | diet | Lycopene | β-carotene | carotene | Phytofluene | Phytoene | 1,5-diol |

FEMALE

| | | | | | | | |
|---|---|---|---|---|---|---|---|
| 1 | 2.5 | 120 | 11 | 17 | 106 | 66 | 9 |
| 2 | 1.0 | 64 | 6 | 8 | 48 | 35 | 5 |
| 3 | 0.5 | 66 | 8 | 7 | 50 | 40 | 7 |
| 4 | 0.25 | 49 | 7 | 2 | 46 | 46 | 4 |
| 5 | 0.10 | 42 | 4 | 4 | 33 | 38 | 6 |
| 6 | 0 | 0 | 0 | 0 | 0 | 0 | 0 |

MALE

| | | | | | | | |
|---|---|---|---|---|---|---|---|
| 1 | 2.5 | 33 | 3 | 5 | 30 | 23 | 4 |
| 2 | 1.0 | 5 | 1 | 1 | 5 | 4 | .39 |
| 3 | 0.5 | 60 | 5 | 5 | 34 | 21 | 4 |
| 4 | 0.25 | 3 | 1 | 1 | — | — | .28 |
| 5 | 0.10 | 12 | 1 | 1 | 10 | 10 | .34 |
| 6 | 0 | 0 | 0 | 0 | 0 | 0 | 0 |

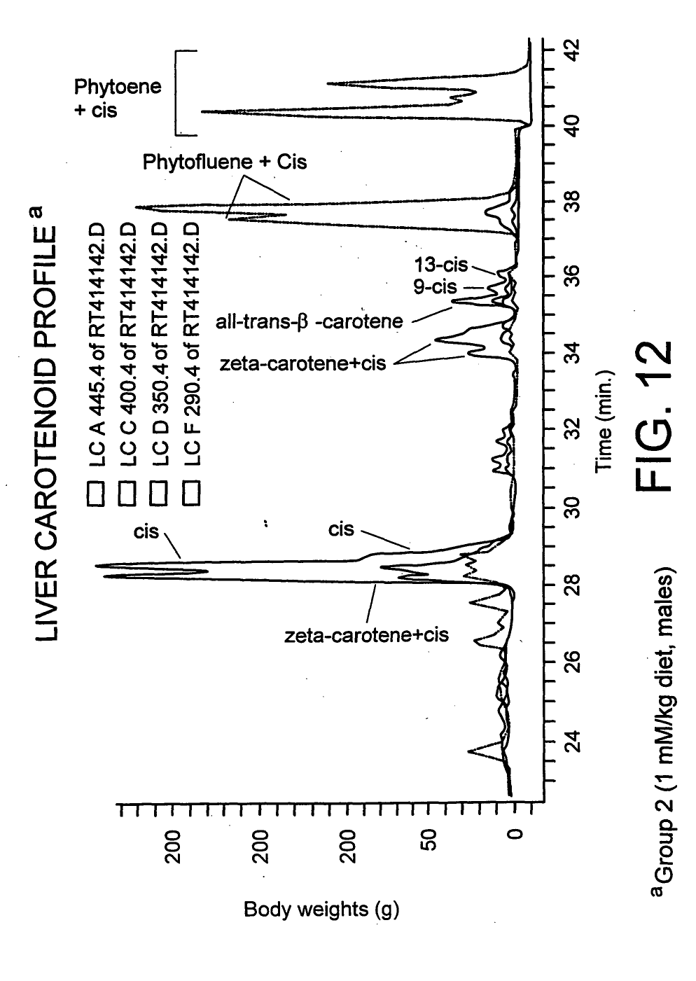

The saponified organic extracts from livers, examined by HPLC-MS, indicated the presence of significant amounts of lycopene, phytoflu ne and phytoene and their geometrical isomers (FIG. 12). In addition, lower levels of zeta-carotene, all trans-β-carotene, 9-cis-β-carotene and 13 cis-β-carotene were also detected.

Two oxidative metabolites of lycopene, which have been previously identified in human serum, namely 2,6 cyclolycopene-1,5-diols I & II and their epoxide precursors, 2,6-cyclolycopene-1,5-epoxide I & II which have not been detected in human serum, were also detected in the liver samples Quantitation and identification of the former metabolites suggests that, in addition to the dietary source of 2,6-cyclolycopene-1-5 diol, the liver actively metabolizes lycopene to these metabolites as well. The amount of lycopene in female livers was higher, in general, than males and a non-linear dose-response curve was exhibited in males but not females. These results indicate that carotenoids present in the Lycopene Suspension are absorbed and stored in the liver and that lycopene is metabolized in the rat in a manner similar to that found in humans.

Serum

Serum lycopene levels ranged from a low of 80 to a high of 370 ng/ml. The lycopene concentration in the serum of supplemented animals varied in a non-linear manner with regard to dose in both males and females (Table 15). Surprisingly, the highest concentrations in females were found in groups 3 (200 ppm) and 4 (100 ppm) rather than group 1 (1000 ppm) as expected. Similarly, among males, the highest concentration was in group 3. In females, the low lycopene (40 ppm) group exhibited the lowest concentration of serum lycopene but in males there was no difference between the highest and lowest lycopene groups.

TABLE 15
LYCOPENE CONTENT OF RAT SERUM (ng/ml)^{a}

| Group No. | \overline{x} ± SD | Median | Range |
|---|---|---|---|
| FEMALE | | | |
| 1 | 187 ± 043 | 205 | 123-232 |
| 2 | 169 ± 042 | 180 | 109-211 |
| 3 | 245 ± 083 | 210 | 174-366 |
| 4 | 313 ± 047 | 308 | 262-369 |
| 5 | 145 ± 053 | 152 | 081-207 |
| 6 | 0 | 0 | 0 |
| MALE | | | |
| 1 | 168 ± 036 | 160 | 134-230 |
| 2 | 227 ± 068 | 225 | 148-326 |
| 3 | 278 ± 066 | 285 | 174-372 |
| 4 | 231 ± 064 | 215 | 153-328 |
| 5 | 171 ± 049 | 177 | 100-238 |
| 6 | 0 | 0 | 0 |

^{a}N = 6
These results suggest that serum lycopene levels are regulated by a homeostatic mechanism involving hepatic storage metabolism and release in a manner similar to vitamin A. The serum levels reported here, in rats, are well within the range reported for humans consuming normal levels of tomatoes, or tomato products, i.e., 0.1-5 μg/ml serum (100-5000 ngs/ml).

Mammary Gland

Lycopene concentrations in the mammary fat pad of female rats ranged from a low of 139 to a high of 460 ng/g wet weight (Table 16). In contrast to serum, a generalized dose-response effect was seen in mammary tissue with respect to dietary lycopene intake (Table 4).

TABLE 16
LYCOPENE CONTENT OF RAT MAMMARY GLAND (ng/g)^{a}

| Group No. | \overline{x} ± SD | Median | Range |
|---|---|---|---|
| 1 | 309 ± 131 | 235 | 232-460 |
| 2 | 200 ± 030 | 197 | 172-231 |
| 3 | 215 ± 062 | 220 | 139-282 |
| 4 | 229 ± 054 | 217 | 181-288 |
| 5 | 174 ± 057 | 143 | 139-239 |
| 6 | 0 | 0 | 0 |

¹N = 3

Prostate

Lycopene concentrations in the prostate gland of male rats ranged from a low of 32 to a high of 147 ng/g wet weight (Table 17). On average, the concentration of lycopene in prostate tissue was an order of magnitude lower than mammary tissue. A dose-related pattern of uptake into the prostate was apparent (Table 17).

TABLE 17
LYCOPENE CONTENT OF RAT PROSTATE GLAND (ng/g)^{a}

| Group No. | \overline{x} ± SD | Median | Range |
|---|---|---|---|
| 1 | 97 ± 17 | 99 | 79-112 |
| 2 | 95 ± 48 | 83 | 54-147 |
| 3 | 50 ± 37 | 35 | 23-93 |
| 4 | 52 ± 26 | 52 | 26-77 |
| 5 | 47 ± 16 | 47 | 32-63 |
| 6 | 0 | | |

^{a}N = 3

Lung

The lycopene content of the lung in male and female rats ranged from a low of 124 to a high of 424 ng/g wet weight (Tables 18 & 19). in the female rat, there appeared to be a plateau effect among groups 1 to 4 followed by a definite decrease in the lowest group (Group 5). In males, a similar result was obtained with the exception of an increase in group 4.

TABLE 18
CONCENTRATION OF LYCOPENE IN LUNG OF MALE RAT

| | Concentration | | Concentration | (ngs/g) |
|---|---|---|---|---|
| Group No. | lycopene in diet | Animal No. | individual | average |
| 1 | 1280 | 36-1 | 193 | 190 |
| | | 36-2 | 187 | |
| 2 | 512 | 41-1 | 170 | 214 |
| | | 41-2 | 257 | |
| 3 | 256 | 46-1 | 325 | 375 |
| | | 46-2 | 424 | |
| 4 | 128 | 51-1 | 239 | 201 |
| | | 51-2 | 162 | |
| 5 | 51 | 56-1 | 167 | 151 |
| | | 56-2 | 135 | |
| 6 | 0 | 61-1 | 0 | 0 |
| | | 61-2 | 0 | |

TABLE 19
CONCENTRATION OF LYCOPENE
IN LUNG OF FEMALE RAT

| | Concentration | | Concentration | (ngs/gm) |
|---|---|---|---|---|
| Group No. | lycopene in diet | Animal No. | individual | average |
| 1 | 1280 | 1-1 | 184 | 227 |
| | | 1-2 | 270 | |
| 2 | 512 | 6-1 | 280 | 246 |
| | | 6-2 | 211 | |
| 3 | 256 | 11-1 | 243 | 193 |
| | | 11-2 | 142 | |
| 4 | 128 | 16-1 | 208 | 211 |
| | | 16-2 | 214 | |
| 5 | 51 | 21-1 | 144 | 134 |
| | | 21-2 | 124 | |
| 6 | 0 | 26-1 | 0 | 0 |
| | | 26-2 | 0 | |

Colon

It was not possible to obtain accurate values for colon tissue because carotenoids entered into the interstices of the mucosa rendering it impossible to assess intracellular (absorbed) carotenoid and luminal (unabsored) carotenoid. In general, the colon values appeared to be similar those of lungs and breast.

Glutathione Assays

The rationale for measuring glutathione is that the reduced form of the tripeptide thiol, glutathione (Glu-Cys-Gly), participates directly in several key intracellular reactions, including protection against free radical damage. In contrast to carotenoids, which reside in the lipid phase, glutathione is found in the aqueous phase of the cell and there is evidence that lipid and aqueous phase anti-oxidants may interact with one another in such a manner that high levels of one may “spare” or limit the deletion of the other. Total (reduced and oxidized) glutathione assays of blood, liver and kidney (Tables 20-22) were conducted.

TABLE 20
WHOLE BLOOD GLUTATHIONE RESULTS - SUMMARY

Glutathione (μmol/g)*

| Lycopene (ppm) | Female | Male |
|---|---|---|
| 0 | 1.010 ± 0.0627 | 0.936 ± 0.0888 |
| 51 | 0.832 ± 0.158⁺ | 1.030 ± 0.0387 |
| 128 | 0.890 ± 0.202 | 0.920 ± 0.169 |
| 256 | 0.848 ± 0.0988 | 0.910 ± 0.106 |
| 512 | 0.930 ± 0.123 | 0.990 ± 0.141 |
| 1280 | 1.080 ± 0.104 | 0.948 ± 0.294 |

*Values are mean ± S.D., n = 5
⁺Significantly different from control, p < 0.05

TABLE 21
LIVER GLUTATHIONE RESULTS - SUMMARY

Glutathione (μmol/g)*

| Lycopene (ppm) | Female | Male |
|---|---|---|
| 0 | 5.45 ± 0.366 | 6.13 ± 0.516 |
| 51 | 6.01 ± 0.309 | 6.77 ± 1.53 |
| 128 | 6.54 ± 1.09⁺ | 5.38 ± 0.363 |
| 256 | 4.87 ± 1.14 | 4.59 ± 1.37 |
| 512 | 5.88 ± 0.492 | 6 .90 ± 1.098 |
| 1280 | 7.38 ± 0.705⁺ | 7.16 ± 1.95⁺ |

*Values are mean ± S.D., n = 5
⁺Significantly different from control, p < 0.05

TABLE 22
KIDNEY GLUTATHIONE RESULTS - SUMMARY

Glutathione (μmol/g)*

| Lycopene (ppm) | Female | Male |
|---|---|---|
| 0 | 0.774 ± 0.154 | 0.592 ± 0.234 |
| 51 | 0.900 ± 0.101 | 0.688 ± 0.316 |
| 128 | 0.950 ± 0.0758 | 0.686 ± 0.170 |
| 256 | 1.030 ± 0.414⁺ | 0.624 ± 0.104 |
| 512 | 1.090 ± 0.163⁺ | 0.854 ± 0.055⁺ |
| 1280 | 0.089 ± 0.045⁺ | 0.828 ± 0.128⁺ |

*Values are mean ± S.D., n = 5
⁺Significantly different from control, p < 0.05

The determination of glutathione was by HPLC with dual electrochemical detection (see Kleinman, W. A. & Richie, J. P., J. Chromatogr B 672:73-80, 1995). only 1 case, at the highest lycopene level, was there a significant increase in glutathione levels and this occurred only in the liver. No dose-related changes in glutathione were noted in liver, kidney or whole blood.

Liver Vitamin A and Vitamin E Assays

The effect of the tomato carotenoid supplement on the absorption and storage of two important fat soluble vitamins, namely, retinol and α-tocopherol, was investigated. Using established HPLC methods, a strong liner dose-response relationship was found between the lycopene content of the liver and both retinol and a-tocopherol concentration in female R-344 rats (Table 23).

TABLE 23
CONCENTRATION OF RETINOL AND
α-TOCOPHEROL IN RAT LIVER

| | | | Liver | |
|---|---|---|---|---|
| | Lycopene in diet | | retinol | |
| Group No. | mM/kg Diet | mg/kg Diet | (μg/gm) | α-tocopherol (μg/gm) |
| Female | | | | |
| 1 | 2.5 | 1280 | 1302 | 47 |
| 2 | 1.0 | 512 | 768 | 38 |
| 3 | 0.5 | 256 | 858 | 39 |
| 4 | 0.25 | 128 | 888 | 46 |
| 5 | 0.10 | 51 | 555 | 32 |
| 6 | 0 | 0 | 120 | 13 |
| Male | | | | |
| 7 | 2.5 | 1280 | 388 | 28 |
| 8 | 1.0 | 512 | 119 | 8 |
| 9 | 0.5 | 256 | 584 | 23 |
| 10 | 0.25 | 128 | — | — |
| 11 | 0.10 | 51 | 454 | 14 |
| Control | 0 | 0 | 16 | 0 |

In the case of retinol there was a 10-fold difference between controls and the highest lycopene dose; in the case of α-tocopherol there was a 4-fold difference. The data from males were similar. Since the supplement contained little or no retinol vitamin E, all of the retinol and vitamins found in the liver must have come from the AIN-76A diet. Hence, the presence of the carotenoid supplement altered the hepatic uptake and storage of both vitamin A and vitamin E.

### SUMMARY

(1) The Lycopene Suspension derived from tomatoes consisted of lycopene as the major constituent (70%) followed by a number of other carotenoids including α- and β-carotene, zeta-carotene, lutein, phytoene and phytofluene.

(2) At the dosage range used, lycopene exerted no deleterious effect on food intake or weight gain in either male or female rats.

(3) Lycopene was stable in the diet for 7 days at ambient light and temperature and 21 days in the dark at 4° C.

(4) Approximately 55% of estimated lycopene consumed per day was excreted in the feces.

(5) Lycopene was readily absorbed and stored in the rat liver. Lycopene was also metabolized in the liver and the metabolic pathway appears to be similar to that found in humans. Lycopene levels in serum were not related in a linear manner with dietary intake, indicating a relatively tight homeostatic control of serum lycopene levels. The concentration of lycopene in serum was 2 orders of magnitude less than that seen in liver. Lycopene was detected in ng amounts in lung, prostate, colon and mammary tissue. In general, uptake was dose-related with the lowest concentrations usually found in the group fed the lowest between males and females, particularly in liver lycopene concentrations.

(6) With the exception of the livers obtained from animals fed the highest lycopene levels, glutathione concentrations in whole blood, liver and kidney were similar to controls in both males and females.

(7) Liver concentrations of Vitamin E and Vitamin A were altered by the intake of the tomato carotenoid supplement in a dose-related manner. Since neither Vitamin E or A were pres nt in the supplement, the increased levels found in the liver involved selective uptake from the diet.

In conclusion, it is clear that in the F-344 rat the entire carotenoid profile in the Lycopene Suspension incorporated into the AIN-76A semipurified diet, is readily absorbed, enters the circulation, and is deposited in all tissues analyzed. The major part of the dietary lycopene absorbed is stored in the liver where it undergoes metabolism in a manner similar to that found in humans. There is no evidence of lycopene toxicity at the dose range used.

While the invention has been described and illustrated herein by references to various specific materials, procedures and examples, it is understood that the invention is not restricted to the particular materials, combinations of materials, and procedures selected for that purpose. Numerous variations of such details can be implied and will be appreciated by those skilled in the art.

What is claimed is:

1. A carotenoid composition comprising lycopene suspended or dispersed in a medium-chain triglyceride.

2. A composition according to claim 1, wherein the medium chain triglyceride is derived from the esterification of a medium chain fatty acid and glycerol.

3. A composition according to claim 2, wherein the fatty acid is a C6 to C12 fatty acid.

4. A composition according to claim 3, wherein the fatty acid is a C8 to C10 fatty acid.

5. A composition according to claim 2, wherein the fatty acid is selected from the group consisting of caprylic acid, capric acid, lauric acid, and mixtures thereof.

6. A composition according to claim 1, comprising up to 10% by weight lycopene.

7. A composition according to claim 6, comprising between 4% and 5% by weight lycopene.

8. A composition according to claim 6, comprising up to 98% by weight medium chain triglyceride.

9. A composition according to claim 8, comprising between 20% and 80% medium chain triglyceride.

10. A composition according to claim 9, comprising between 30% and 70% by weight medium chain triglyceride.

11. A composition according to claim 8, further comprising an oil-soluble antioxidant.

12. A composition according to claim 11, wherein the oil-soluble anti-oxidant is selected from the group consisting of tocopherols, butylated hydroxy anisole, butylated hydroxytoluene, propyl gallate, ethoxyquin and ascorbyl palmitate, natural tocopherols, and mixtures thereof.

13. A composition according to claim 12, wherein the tocopherol is selected from the group consisting of beta, gamma-, and delta-tocopherols and mixtures thereof.

14. A composition according to claim 11, wherein the anti-oxidant comprises up to 15% by weight of the composition.

15. A composition according to claim 14, wherein the anti-oxidant comprises between 5% and 10% by weight of the formulation.

16. A method of providing a bioavailable antioxidant comprising orally administering to a host an effective amount of lycopene suspended or dispersed in a medium chain triglyceride.
